(19) United States
(12) Patent Application Publication
(10) Pub. No.: US 2026/0216440 A1
(43) Pub. Date: Jul. 30, 2026

(54) Medical or pharmaceutical stopper for sealing a container volume and method for manufacturing the same

(71) Applicant: RAUMEDIC AG, Münchberg (DE)

(72) Inventors: Jörg PRESCHER, Ramsthal (DE); Daniel MORETH, Hof (DE)

(21) Appl. No.: 19/150,640

(22) PCT Filed: Jan. 24, 2024
(86) PCT No.: PCT/EP2024/051605

§ 371 Date: Jul. 24, 2025

(30) Foreign Application Priority Data
Jan. 24, 2023 (DE) 10 2023 200 491.7

(51) Int. Cl. A61M 5/315

(52) U.S. Cl. CPC A61M 5/31505; A61M 2005/31506; A61M 2205/0216; A61M 2205/0238; A61M 2207/10

(57) ABSTRACT

A medical or pharmaceutical stopper (3) is used to seal the container volume of a container for receiving a liquid (5) for medical, pharmaceutical, or analytical use. The stopper has a stopper base body (6) made of a base body material and a stopper overmold section (7) made of an overmolding material, via which the stopper (3) can be sealingly placed on an inner wall (8) of the container. A method for manufacturing an embodiment of the stopper comprising a stopper pre-mold body, a stopper mold-on body, and an at least partially overmolded barrier laminate, is additionally provided. The result is a medical stopper with an improved practical value.

The invention relates to a medical or pharmaceutical stopper for sealing a container volume for receiving a liquid for medical, pharmaceutical, or analytical use. The invention also relates to a method manufacturing the same.

A medical stopper adapted as a closure stopper of the type mentioned above is known from EP 2 985 015 B1. Medical stoppers in the form of syringe plungers are also known.

It is an object of the invention to further develop a medical or pharmaceutical stopper of the type mentioned above such as to improve its practical value.

This object is solved by the invention by providing a medical or pharmaceutical stopper comprising the features specified in claim 1.

The medical or pharmaceutical stopper may be configured as a syringe plunger, i.e., as a stopper moveable relative to an inner wall of a container, or as a closure stopper, which at least partially closes a container in a stationary manner. The liquid for the medical, pharmaceutical, or analytical use may be a pharmaceutical or a blood thinner contained in the container volume to be sealed.

Designing the stopper with a stopper base body and a stopper overmold section molded around the stopper base body makes it possible to select a different overmolding material for the stopper overmold section than for the base body material. The materials for the base body material on the one hand and the overmolding material on the other hand can then be selected largely independently of one another in order to optimize the properties of the medical stopper. The base body material may, for example, be adapted for optimizing a barrier with respect to gases, liquids, and/or solids, e.g. powder material, such as to optimize a barrier function of the stopper. In particular, it is thus possible to avoid using natural rubber or rubber as a material for the stopper base body, as these materials have disadvantages in handling. The overmolding material may in turn be adapted for optimizing the sealing and/or sliding properties of the stopper such as to optimize its sealing and/or sliding function. Due to the additional stopper overmold section, it is possible to avoid in particular silicone oil wetting of the stopper base body, since the properties that would otherwise be achieved by such silicone oil wetting can thus be achieved by means of the stopper overmold section. All things considered, this results in a stopper with a high practical value.

Regarding a stopper according to claim 2, the stopper base body is suitable for mass manufacturing. In addition, the base body material can be selected with respect to high barrier properties.

Base body materials according to claim 3 have proven particularly effective in practice.

Alternatively, according to claim 4, silicone may also be used as the base body material for the stopper base body.

Selecting silicone also for the overmolding material for the stopper overmold section according to claim 5 may thus provide a medical stopper which, in particular, prevents an undesirable stick-slip effect when the stopper abuts on the inner wall of the container. The silicone selected as the overmolding material can be optimized in particular for good sealing and/or sliding properties.

Connections between the stopper base body and the stopper overmold section according to claims 6 and 7, respectively, have proven their worth in practice. A mechanical connection between the stopper base body and the stopper overmold section may be achieved by means of form fit, in particular an undercut and/or a snap-fit. A chemical connection between the stopper base body and the stopper overmold section can be achieved by adhesion.

A receptacle of the stopper base body according to claim 8 improves the function thereof when the medical stopper is adapted as a syringe plunger. The receptacle for an end section of the plunger rod may be configured as a simple opening, as a conical receptacle or as a threaded receptacle.

In an embodiment of the stopper overmold section according to claim 9, a barrier property of the stopper overmold section is improved by the barrier laminate. The barrier properties of the barrier laminate may be gas barrier properties, liquid barrier properties or solid barrier properties, for example regarding the suitability of a barrier for powder material. The overmolding material and also the base body material of the medical stopper can then have lower barrier properties, which further simplifies the choice of materials for these materials, or facilitates other functional optimizations. In particular, when using a stopper overmold section with a barrier laminate, the overmolding material may be made of a silicone material. The barrier laminate may be a film. The laminate material may be at least one of the following materials: a metal, a metal alloy, glass, PE, PC, PMMA, PBT, PA, PEEK. The barrier laminate may cover at least 40%, at least 50%, at least 60%, at least 70%, at least 80%, or at least 90% of the sealing cross-section of the stopper. The barrier laminate may also cover the entire cross-section of the stopper. The barrier laminate may have at least one opening by means of which the stopper pre-mold body is in direct contact with the stopper mold-on body. The stopper pre-mold body may have at least some sections having a shape complementary to the laminate. The barrier laminate may be partially exposed, in particular on the side of the medical stopper facing away from the liquid. The sealing cross-section of the stopper is defined by the stopper cross-section that can be sealingly placed against the inner wall of the container.

The advantages of a manufacturing method according to claim 10 correspond to those already explained above in connection with the medical stopper, in particular according to claim 9.

When the barrier laminate according to claim 11 is completely overmolded, it may also be made of less biocompatible materials, which again facilitates the selection of the laminate material.

A tempering process according to claim 12 allows for cross-linking at least one stopper material or degassing at least one stopper material, if necessary.

In particular, a durable medical stopper can be provided. The stopper may be configured such as to be very biocompatible. The stopper may be configured such as to be sterilizable with at least one common sterilization method, for example sterilization with gamma or X-rays, electron beams, autoclaving, or treatment with ethylene oxide.

The entire stopper can be manufactured by injection-molding. The resulting stopper can have very good barrier properties.

Example embodiments of the invention are explained in more detail below with reference to the drawings. In the drawings:

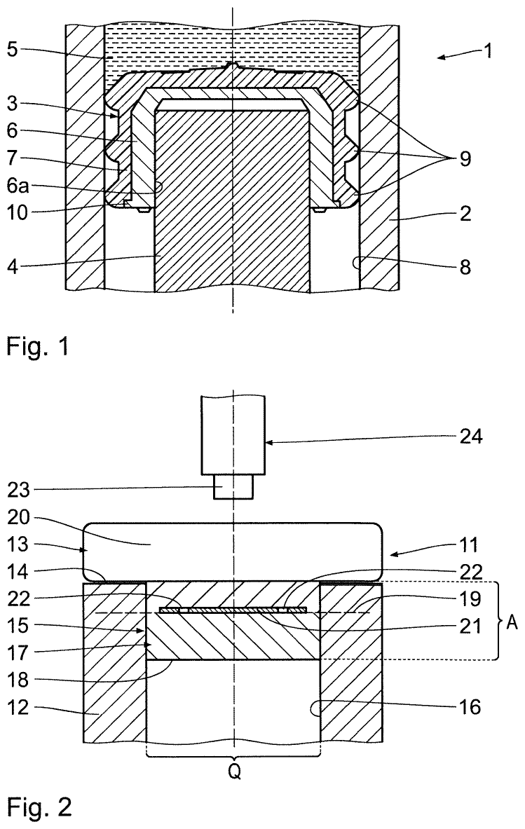

FIG. 1 shows a cross-section of a medical stopper for sealing a container volume for receiving a liquid for medical, pharmaceutical, or analytical use, wherein the stopper is adapted as a movable stopper in the form of a syringe plunger;

FIG. 2 shows, partly in cross-section, a further embodiment of a medical stopper for sealing a container volume for receiving a liquid for medical, pharmaceutical, or analytical use, wherein the stopper is adapted as a closure stopper for a liquid container;

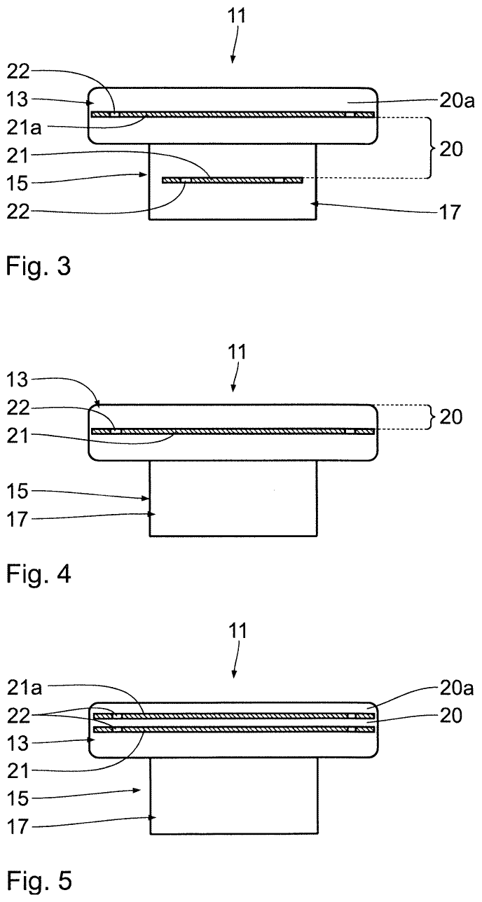

FIGS. 3 to 5 show in an illustration similar to FIG. 2, further embodiments of a medical stopper with other arrangement variants of at least one overmolded barrier laminate than in the embodiment according to FIG. 2;

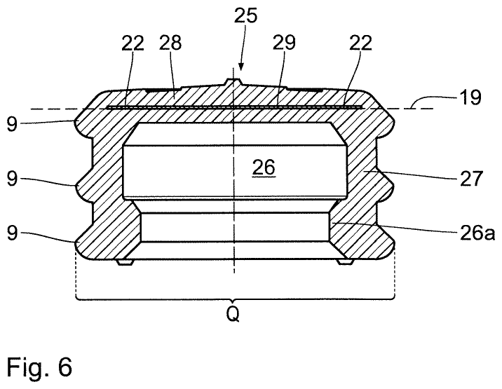

FIG. 6 shows in an illustration similar to FIG. 1, a further embodiment of the medical stopper in the form of a syringe plunger;

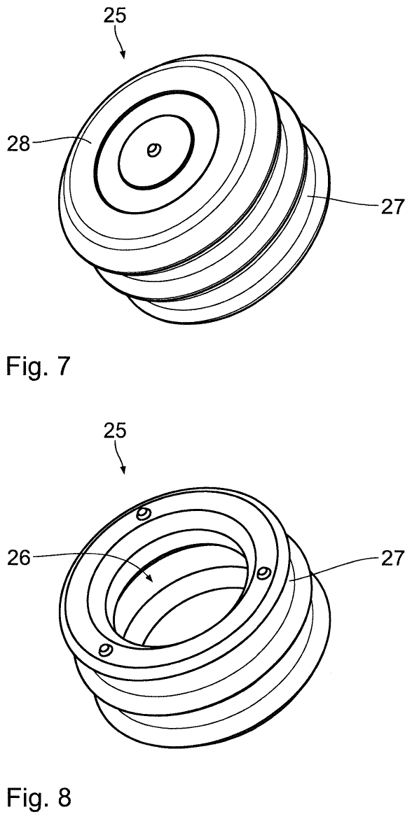

FIG. 7 shows a perspective view of the medical stopper according to FIG. 6;

FIG. 8 shows a perspective view of the stopper according to FIG. 7, seen from the opposite direction compared to FIG. 7;

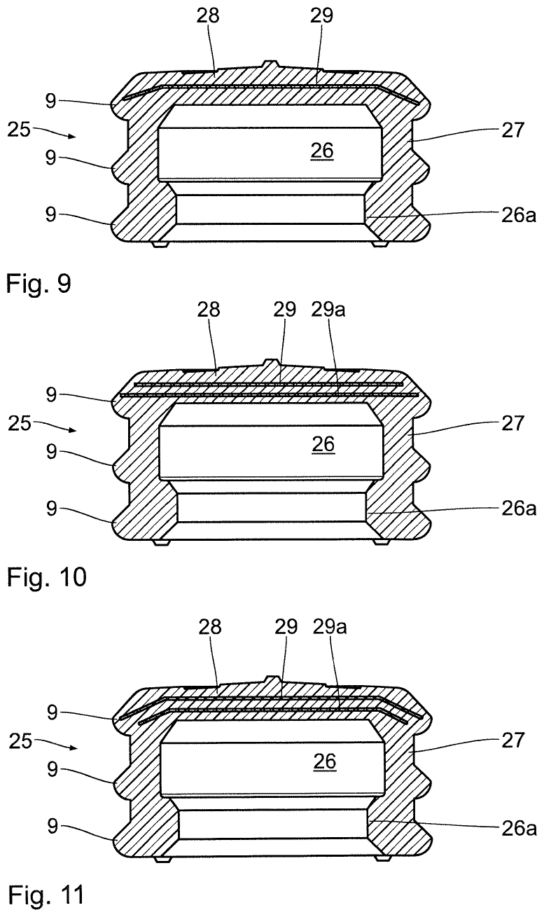

FIGS. 9 to 11 show in an illustration similar to FIG. 6, the syringe plunger with further arrangement variants of at least one overmolded barrier laminate;

FIGS. 12 and 13 show in an illustration similar to FIGS. 7 and 8, a further embodiment of a medical stopper;

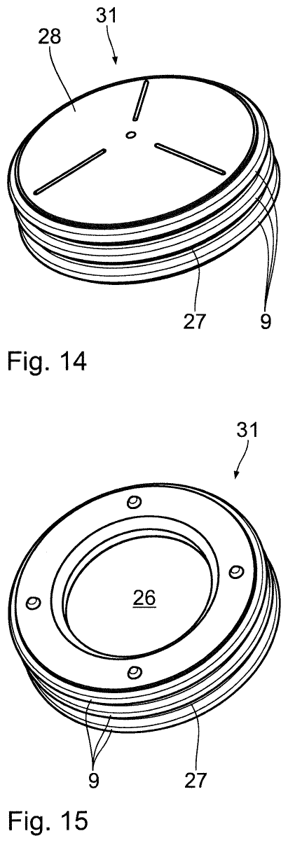

FIGS. 14 and 15 show in an illustration similar to FIGS. 7 and 8, a further embodiment of a medical stopper;

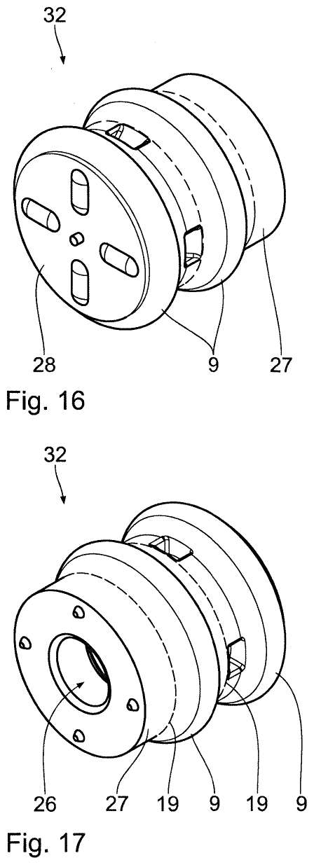

FIGS. 16 and 17 show in an illustration similar to FIGS. 7 and 8, a further embodiment of a medical stopper;

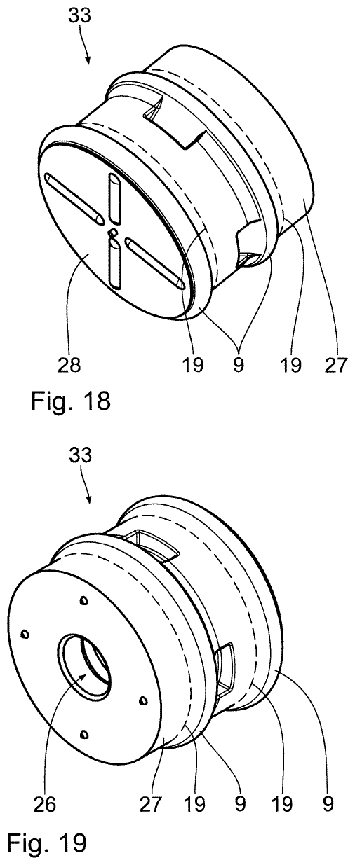

FIGS. 18 and 19 show in an illustration similar to FIGS. 7 and 8, a further embodiment of a medical stopper;

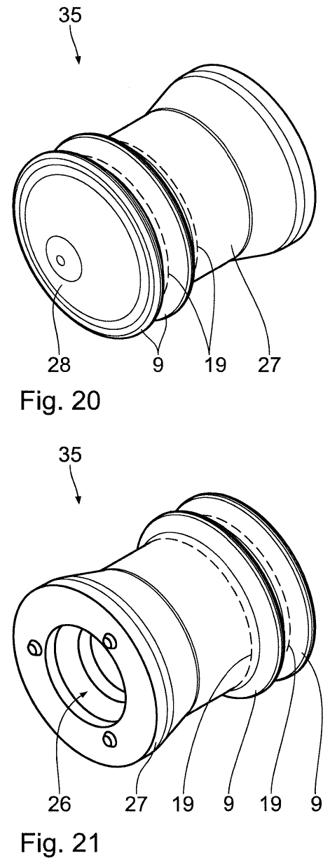

FIGS. 20 and 21 show in an illustration similar to those shown in FIGS. 7 and 8, a further embodiment of a medical stopper;

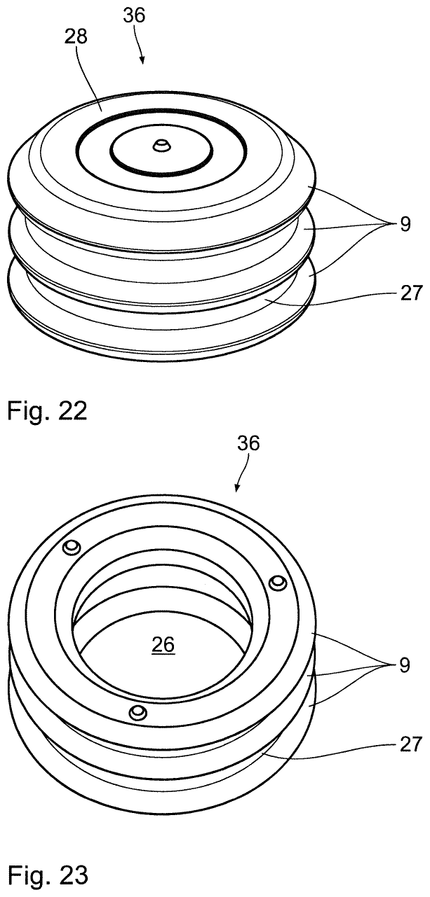

FIGS. 22 and 23 show in an illustration similar to FIGS. 7 and 8, a further embodiment of a medical stopper;

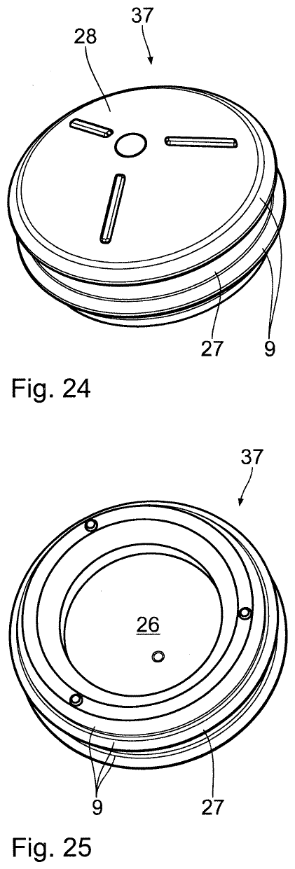

FIGS. 24 and 25 show in an illustration similar to FIGS. 7 and 8, a further embodiment of a medical stopper;

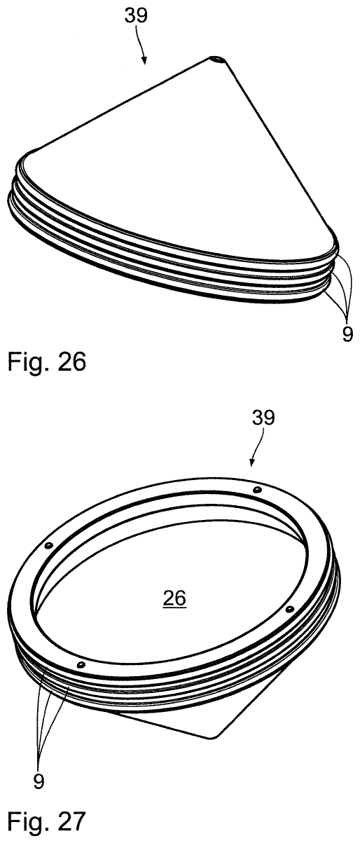

FIGS. 26 and 27 show in an illustration similar to FIGS. 7 and 8, a further embodiment of a medical stopper;

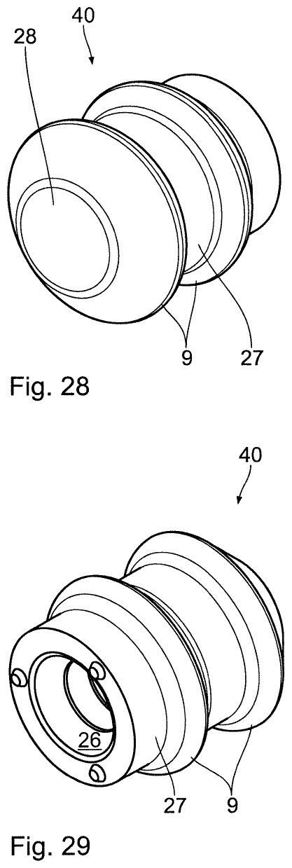

FIGS. 28 and 29 show in an illustration similar to FIGS. 7 and 8, a further embodiment of a medical stopper;

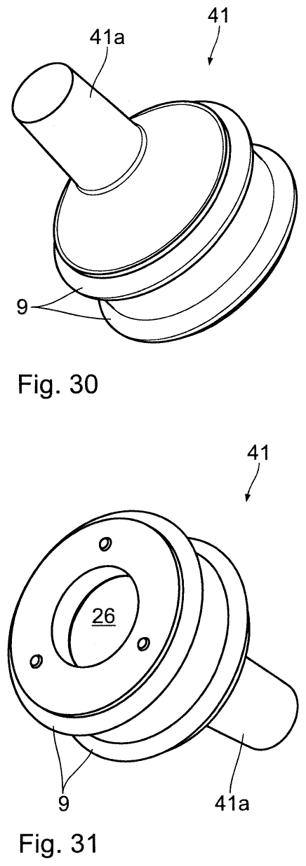

FIGS. 30 and 31 show in an illustration similar to FIGS. 7 and 8, a further embodiment of a medical stopper;

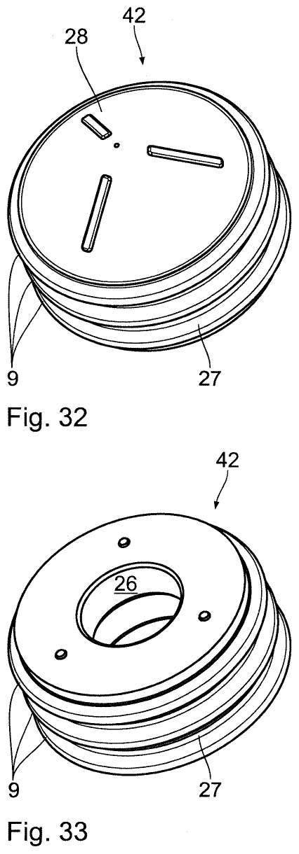

FIGS. 32 and 33 show in an illustration similar to FIGS. 7 and 8, a further embodiment of a medical stopper;

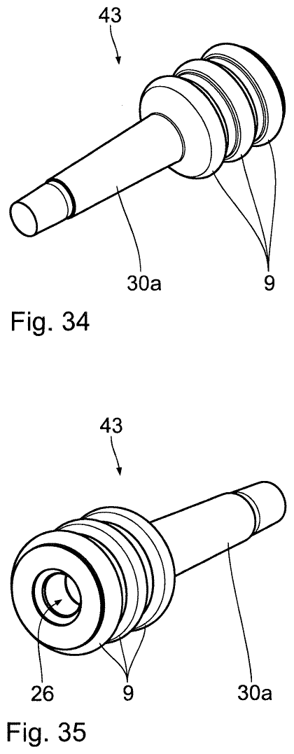

FIGS. 34 and 35 show in an illustration similar to FIGS. 7 and 8, a further embodiment of a medical stopper;

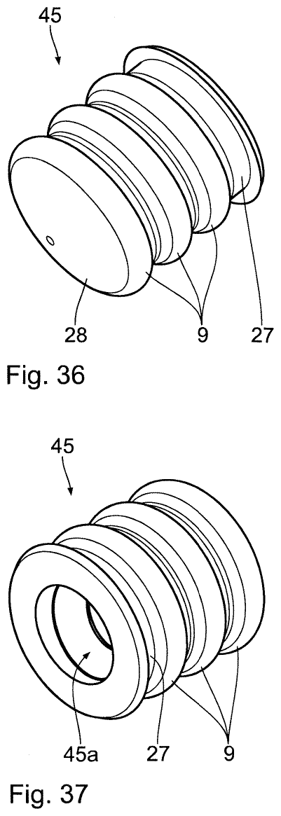

FIGS. 36 and 37 show in an illustration similar to FIGS. 7 and 8, a further embodiment of a medical stopper;

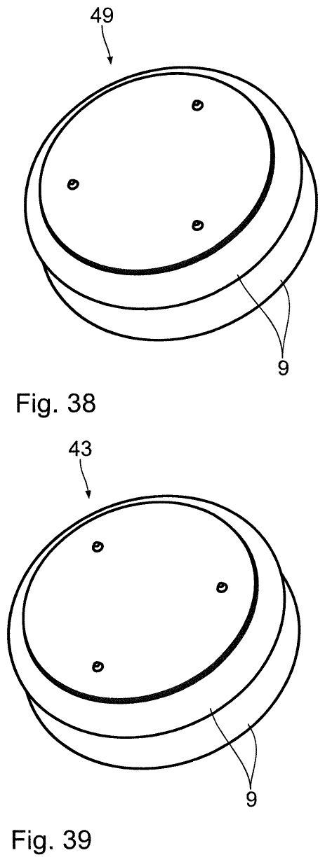

FIGS. 38 and 39 show in an illustration similar to FIGS. 7 and 8, a further embodiment of a medical stopper;

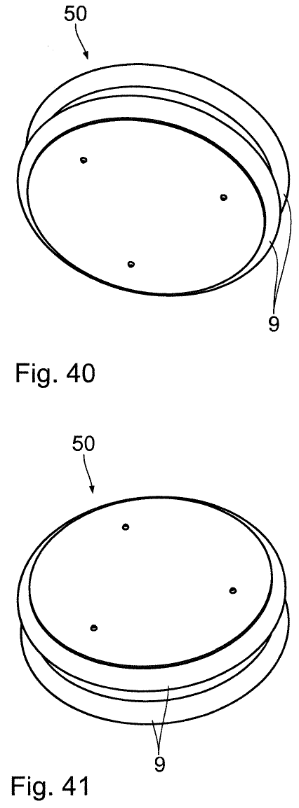

FIGS. 40 and 41 show in an illustration similar to FIGS. 7 and 8, a further embodiment of a medical stopper;

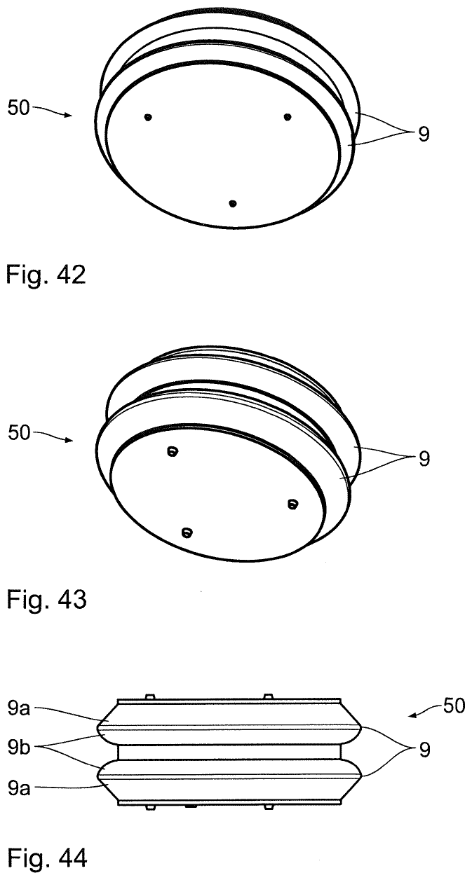

FIGS. 42 and 43 show in an illustration similar to FIGS. 7 and 8, a further embodiment of a medical stopper;

FIG. 44 shows a side view of the stopper according to FIGS. 42 and 43;

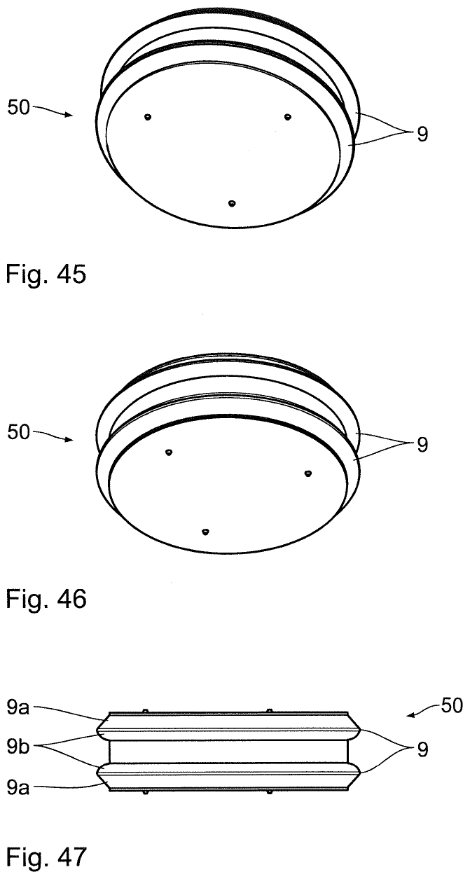

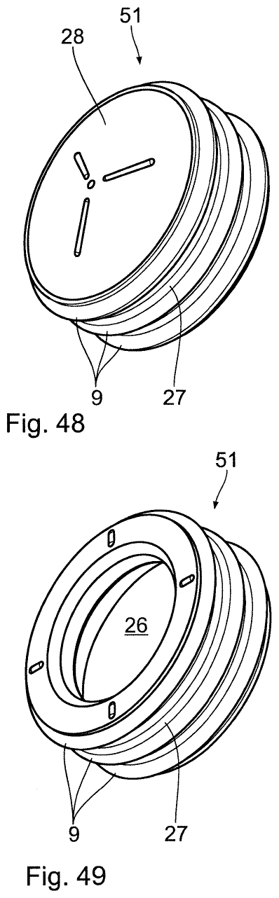

FIGS. 45 to 47 show in an illustration similar to FIGS. 42 to 44, a further embodiment of a medical stopper;

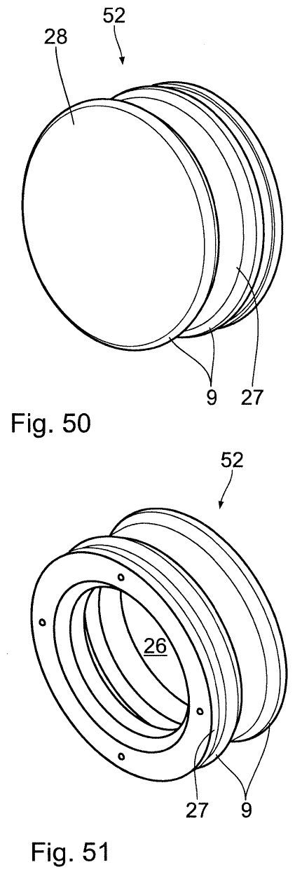

FIGS. 48 and 49 show in an illustration similar to FIGS. 7 and 8, a further embodiment of a medical stopper;

FIGS. 50 and 51 show in an illustration similar to FIGS. 7 and 8, a further embodiment of a medical stopper; and

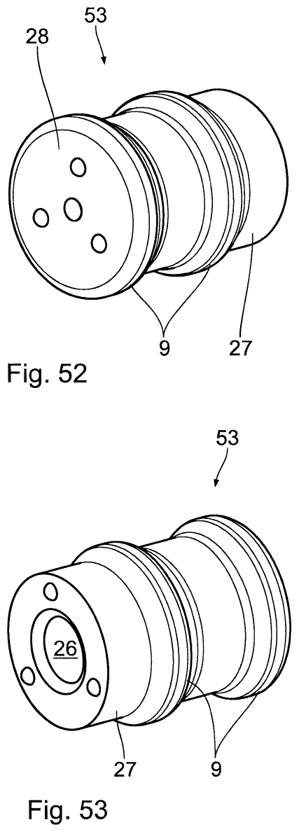

FIGS. 52 and 53 show in an illustration similar to FIGS. 7 and 8, a further embodiment of a medical stopper.

FIG. 1 shows, in an axial longitudinal section, a section of a pre-filled syringe 1 for storing and subsequently dispensing a liquid for medical, pharmaceutical, or analytical use. The syringe 1 has, as a container for the liquid, a syringe cylinder 2, in which a medical stopper in the form of a plunger 3 is movably guided. The plunger 3 is connected to a plunger rod 4 of the syringe 1, the plunger rod 4 being only partly shown in FIG. 1. Depending on the embodiment of the plunger 3, such plunger rod may also be omitted.

The plunger 3 serves to seal the syringe cylinder 2, i.e. to seal a container volume for receiving the liquid, which is in FIG. 1 implied as 5. The liquid may be a blood thinner or a pharmaceutical.

The plunger 3 comprises a stopper base body 6 made of a base body material and a stopper overmold section 7 made of an overmolding material. The stopper base body 6 has a receptacle 6a for a section of the plunger rod 4. The receptacle 6a may be configured as a simple opening, in the form of a thread, or, for example, in a conical shape.

The plunger 3 is in its entirety an injection-molded part manufactured by a two-component injection-molding process.

The plunger 3 abuts, via the stopper overmold section 7, sealingly against an inner wall 8 of the plunger cylinder 2. For this purpose, the stopper overmold section 7 comprises several, in the present case three, sealing lips 9 made of the overmolding material, which are axially spaced apart from one another and abut on the inner wall 8. Depending on the embodiment of the plunger 3, two sealing lips, four sealing lips or even more sealing lips may be provided. An embodiment with exactly one sealing lip is also possible.

When the plunger 3 is used, the sealing lips 9 slide relative to the inner wall 8.

The base body material of the stopper base body 6 is a thermoplastic and, in particular, at least one of the following materials: PC (polycarbonate), PMMA (polymethyl methacrylate), PBT (polybutylene terephthalate), PA (polyamide) or PEEK (polyether ether ketone). The base body material can be in particular optimized for a barrier function and may have a gas permeability that is lower than that of natural rubber, for example 90%, 75%, 60%, 50%, 40%, 30%, 20%, 10% of the gas permeability of natural rubber, or an even lower gas permeability compared to natural rubber.

As an alternative to a thermoplastic, the base body material of the stopper base body 6 may also be a silicone.

The overmolding material of the stopper overmold section 7 is a silicone material, in particular LSR (liquid silicone rubber), HCR (high consistency rubber) silicone, i.e., solid silicone rubber, and/or high-temperature crosslinking silicone rubber (HTV).

The overmolding material can be optimized such as to have good sliding properties.

When measuring sliding properties in accordance with ISO 7886/1, the overmolding material has a sliding friction and/or a breakaway force that is improved by at least 10%, at least 20% and, in particular, at least 25% compared to standard test specimens made of pharmaceutical rubber having the same geometry and dimensions.

Depending on the material combination of the base body material of the stopper base body 6 and the overmolding material of the stopper overmold section 7, these two components 6, 7 are mechanically and/or chemically connected to one another, i.e., in particular by adhesion. A mechanical connection may be provided via an undercut 10, e.g. a circumferential collar shown as an example for the plunger 3, which collar is formed on the stopper base body 6 made from the base body material and around which the overmolding material of the stopper overmold section 7 is injection-molded.

The stopper overmold section 7 surrounds the stopper base body 6 radially, i.e., toward the inner wall 8 of the syringe cylinder 2, and also axially upward in FIG. 1, i.e., toward the liquid 5. The stopper base body 6 therefore does not come into contact with the liquid 5 at any point when the plunger 3 is in use.

When injection molding the plunger 3, the stopper base body 6 is molded first. Subsequently, it is overmolded by the stopper overmold section 7.

FIG. 2 shows another embodiment of a medical stopper in the form of a closure stopper 11 for sealing a container volume for receiving a liquid for medical, pharmaceutical, or analytical use.

FIG. 2 shows the closure stopper 11 inserted into a container neck 12 of a not further illustrated container for storing the liquid. The closure stopper 11 has a radially protruding collar section 13 placed on a front wall 14 of the container neck 12, and a stopper section 15 that is inserted into the container neck 12 from the front wall side like a wine cork and seals radially against an inner wall 16 of the container neck 12. The stopper section 15 merges into the collar section 13 with a circumferential step.

The closure stopper 11 has a stopper pre-mold body 17 in the form of a stopper disc with an axial extension A between a free front face 18 of the closure stopper 11 and a connecting plane 19. The stopper pre-mold body 17 forms an axial section of the stopper section 15. A stopper mold-on body 20 is injection-molded onto the stopper pre-mold body 17. The stopper mold-on body 20 comprises the remaining stopper section 15 and the collar section 13 of the closure stopper 11.

The stopper pre-mold body 17 is made of a pre-mold material. The stopper mold-on body 20 is made of a mold-on material. Depending on the embodiment of the closure stopper 11, the pre-mold material and the mold-on material may be the same material or different materials.

A barrier laminate 21 made of a laminate material is laminated onto the stopper pre-mold body 17 in the area of the connection plane 19. The barrier laminate 21 may be a film. The barrier laminate 21 is arranged between the stopper pre-mold body 17 and the stopper mold-on body 20 and is entirely overmolded by the stopper pre-mold body 17 and the stopper mold-on body 20.

The laminate material of the barrier laminate 21 has barrier properties with respect to at least one gas and/or with respect to at least one liquid and/or with respect to at least one solid material, in particular in powder form. Accordingly, the barrier laminate 21 serves as a barrier for the corresponding gas and/or the corresponding liquid and/or the corresponding solid.

The barrier laminate 21 covers at least 30% of a sealing cross-section Q of the closure stopper 11, with Q corresponding to the inner diameter of the container neck 12 in the area of the stopper section 15. An outer diameter of the stopper section 15 is, in a force-free state i.e., when the closure stopper 11 has not yet been inserted into the container 12, slightly larger than the inner diameter of the container neck 12.

The pre-mold material of the stopper pre-mold body 17 and the mold-on material of the stopper mold-on body 20 may be silicone, in particular LSR, HCR or HTV.

The laminate material of the barrier laminate 21 may be a metal and/or a metal alloy and/or a glass and/or a plastic material, in particular a thermoplastic and/or a natural material, for example hemp, an algae material or spider silk. The laminate material may be a woven fabric, for example a woven textile fabric. If the laminate material is a woven fabric, this woven fabric may for example be manufactured by injection molding. The woven fabric may be a coated or an uncoated woven fabric. The barrier laminate 21 may also be manufactured based on a powder and/or sintered material.

Examples of plastic materials may be those mentioned above in connection with the base body material of the stopper base body 6 of the plunger 3 according to FIG. 1. An example of a metal is aluminium. Another example of a plastic material is PE (polyethylene).

The barrier laminate 21 may have at least one opening, through which the stopper pre-mold body 17 is in direct contact with the stopper mold-on body 20. FIG. 2 shows two such openings 22, which are arranged off-center in the barrier laminate 21. Alternatively, the opening in the barrier laminate 21 may be a central opening. Depending on the embodiment of the barrier laminate, it may also be configured without any opening.

The stopper pre-mold body 17 may, at least in some sections, have a shape complementary to the barrier laminate 21. This complementary shape of the stopper pre-mold body 17 can make it easier to position the barrier laminate 21 relative to the stopper pre-mold body 17 before the stopper mold-on body 20 in molded on. Such a complementary shape of the stopper pre-mold body 17 with respect to the barrier laminate 21 may be achieved by raising the stopper pre-mold body 17 at the location of at least one of the laminate openings 22. To position the barrier laminate 21, it is then placed on the already molded stopper pre-mold body 17 such that the respective raised part of the stopper pre-mold body 17 engages in the respective laminate opening 22.

The stopper pre-mold body 17 and the stopper mold-on body 20 can be mechanically and/or chemically connected to one another. The above explanation regarding the connection between the stopper base body 6 and the stopper overmold section 7 of the plunger embodiment shown in FIG. 1 applies here as well.

Such a mechanical connection may generally be achieved by form fit and, for example, also by an undercut or by a snap-fit.

To manufacture the closure stopper 11, first the stopper pre-mold body 17 is manufactured by injection-molding, pressing, casting, gluing, or joining. The barrier laminate is also manufactured and can, for example, be punched out from a flat material.

The barrier laminate 21 is then positioned by placing it on the stopper pre-mold body 17. The stopper mold-on body 20 is then injection-molded onto the stopper pre-mold body 17 such that the barrier laminate 21 is completely overmolded by the stopper pre-mold body 17 and the stopper mold-on body during the mold-on process.

The pre-molding process of the stopper pre-mold body 17 and the mold-on process of the stopper mold-on body 20 onto the stopper pre-mold body 17 may be carried out in the same injection-molding tool, in particular, in different injection cavities of the injection-molding tool.

The injection-molding processes may be followed by a tempering step for cross-linking the injection-molding material and/or degassing the injection-molding material.

For manufacturing the closure stopper 11, a robot may be used to punch out the barrier laminate 21 and/or to load the at least one tool cavity and/or to remove the finished injection-molded closure stopper 11. The finished injection-molded closure stopper 11 may be temporarily packaged before tempering, for which a PE bag may be used. After the tempering step and a final inspection, the manufactured closure stopper 11 may then be packaged according to customer specifications.

If the barrier laminate 21 is provided with a central through-opening, it may be configured such that a tip section 23 of a syringe 24 penetrates this central through-opening 22 when the liquid is removed from the container having the container neck 12, without parts of the barrier laminate 21 being entrained by the tip section 23, which would undesirably detach these parts from the remaining barrier laminate 21.

Depending on the manufacturing method, the stopper pre-mold body and the stopper mold-on body may also be reversed. The body 20 may thus represent the stopper pre-mold body and the body 17 may represent the stopper mold-on body.

Further arrangement variants for the barrier laminate 21 are described below with reference to FIGS. 3 to 5. Components and functions corresponding to those already explained above with reference to FIGS. 1 and 2 are designated with the same reference numbers and are not discussed again in detail.

The stopper 11 according to FIG. 3 comprises, in addition to the barrier laminate 11 in the area of the stopper section 15, another barrier laminate 21a in the collar section 13. In addition to the pre-mold body 17 and a first mold-on body 20 between the two barrier laminates 21, 21a, the stopper 11 according to FIG. 3 comprises another mold-on body 20a which, together with the mold-on body 20, overmolds the barrier laminate 21a. The laminate arrangement according to FIG. 3 can be considered a two-layer barrier laminate 21, 21a, which further improves the barrier properties of the stopper according to FIG. 3 or lowers the requirements for the barrier properties of the individual barrier laminate 21 and 21a, respectively.

In the stopper 11 according to FIG. 4, the barrier laminate 21 is provided in the collar section 13. The pre-mold body 17 is then formed by the entire stopper section 15 and, as shown in FIG. 4, a lower region of the collar section 13. The remaining section of the stopper section 15 is formed by the mold-on body 20, which is, together with the pre-mold body 17, overmolded around the barrier laminate.

In the plunger 11 according to FIG. 5, two barrier laminates 21, 21a are arranged in layers one above another in the collar section 13 of the stopper section. The two barrier laminates 21, 21a may either be laminated directly on top of one another or may again have an mold-on body like the mold-on body 20 of the plunger according to FIG. 3 arranged between them.

FIGS. 6 to 8 describe another embodiment of a medical stopper, again in the form of a syringe plunger 25. Components and functions corresponding to those already explained above with reference to FIGS. 1 and 2 are designated with the same reference numbers and are not discussed again in detail.

A basic structure of the plunger 25 can be considered a synthesis of the stopper base body 6 and the stopper overmold section 7 of the plunger 3, wherein a receptacle 26 for a plunger rod of the type of the plunger rod 4 is, for the plunger 25, formed with an undercut collar 26a.

The plunger 25 does not comprise an overmolded base body made of abase body material, but rather a base body of the plunger 25 is formed—analogous to the embodiment of the closure stopper 11 according to FIG. 2—of a stopper pre-mold body 27, to which a stopper mold-on body 28 is connected on the other side of a connecting plane 19. The stopper mold-on body 28 forms a cap of the plunger 25 facing the volume of the liquid 5 (see FIG. 1).

A barrier laminate 29 is arranged between the stopper pre-mold body 27 and the stopper mold-on body 28 in the area of the connection plane 19. For the stopper pre-mold body 27, the stopper mold-on body 28, and the barrier laminate 29 of the plunger 25, the above description for the stopper pre-mold body 17, the stopper mold-on body 20, and the barrier laminate 21 of the closure plunger 11 applies as well.

When manufacturing the plunger 25, the stopper pre-mold body 27 is premolded and the barrier laminate 29 is, for example manufactured, by punching it out of a flat material. Then the barrier laminate 29 is again positioned such as to abut on the stopper pre-mold body 27 and the stopper mold-on body 28 is injection-molded onto the stopper pre-mold body 27 such that the barrier laminate 29 is completely overmolded by the stopper pre-mold body 27 and the stopper mold-on body 28. The explanation regarding the manufacturing of the closure stopper 11 according to FIG. 2 applies here as well.

The stopper pre-mold body 27 is made of a pre-mold material and the stopper mold-on body 28 is made of a mold-on material. The above explanation regarding the closure stopper 11 applies to these materials as well. The pre-mold material and/or the mold-on material may in particular be silicone, in particular LSR and/or HCR silicone and/or HTV silicone.

The barrier laminate 29 again covers at least 30% of a sealing cross-section Q of the plunger 25.

In an unillustrated embodiment, the plunger 25 may, in addition to the stopper pre-mold body 27 and the stopper mold-on body 28, which together form a stopper overmold section, also comprises a stopper base body made of a base body material, as explained above in connection with FIG. 1.

FIGS. 9 to 11 show further arrangement variants for the barrier laminate 29. Components and functions corresponding to those already explained above with reference to FIGS. 6 to 8 are designated with the same reference numbers and are not discussed again in detail.

In the plunger 25 according to FIG. 9, the barrier laminate 29 radially extends into a radially outer peripheral section and into the uppermost sealing lip 9 shown in FIG. 9. In the arrangement according to FIG. 9, the barrier laminate 29 thus extends practically over the entire outer diameter of the plunger 25. To adapt it to an outer contour of the pre-mold body 27 and the mold-on body 28, a radially outer section of the barrier laminate 29 is angled like a truncated cone section.

In the plunger 25 according to FIG. 10, similar to the arrangement according to FIG. 5, two barrier laminates 29 are located one above one another in the front face section of the plunger 25. The barrier laminate 29a arranged below the barrier laminate 29 in FIG. 10 has such a large radial extension that it extends into the uppermost sealing lip 9 of the plunger 25 according to FIG. 10.

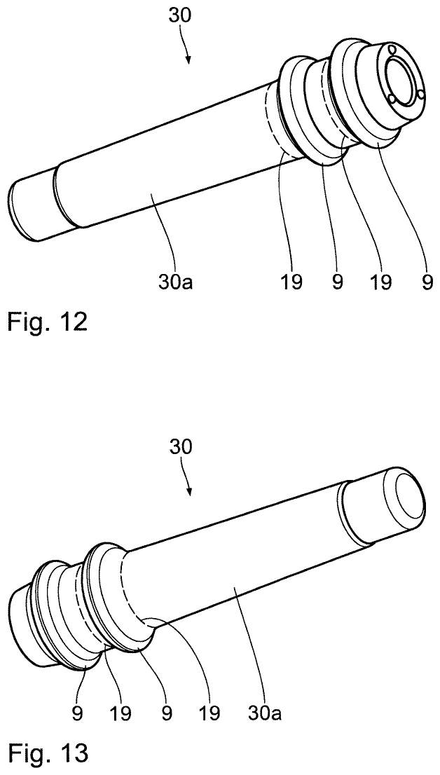

FIG. 11 shows the plunger 25 with an arrangement of two barrier laminates 29, 29a located one above one another like FIG. 10, which are each angled in a truncated cone shape on the circumferential side like in FIG. 9.

Both the radial enlargement of the barrier laminate in the arrangements according to FIGS. 9 to 11 and the use of several barrier laminates 29, 29a located one above one another in the arrangements according to FIGS. 10 and 11 improves either the respective barrier property of the plunger 25 or lowers the requirements for the barrier property of a single barrier laminate 29 or 29a.

FIGS. 12 and 13 show another embodiment of a plunger 30 with an integrated residual discharge peg 30a. Functions and components corresponding to those already explained above with reference to FIGS. 1 to 11 are designated with the same reference numbers and are not discussed again in detail.

The plunger 30 may again be configured with a stopper base body made of a base body material and a stopper overmold section made of an overmolding material, as explained above in connection with FIG. 1. Alternatively, or additionally, the plunger 30 may be configured with a stopper pre-mold body made of a pre-mold material, a stopper mold-on body made of a mold-on material, and an intermediate barrier laminate, as already explained above with reference to FIGS. 2 to 11. Two examples of the connection plane 19 are illustrated in FIG. 6. Other positions of the connection plane 19 between the stopper pre-mold body and the stopper mold-on body are also possible in a corresponding embodiment of the plunger 30.

The plunger 30 comprises two sealing lips 9.

FIGS. 14 and 15 show another embodiment of a plunger 31. Functions and components corresponding to those already explained above with reference to FIGS. 1 to 13 are designated with the same reference numbers and are not discussed again in detail.

The plunger 31 may again be configured with a stopper base body made of a base body material and a stopper overmold section made of an overmolding material, as explained above in connection with FIG. 1. Alternatively, or additionally, the plunger 31 may be configured with a stopper pre-mold body made of a pre-mold material, a stopper mold-on body made of a mold-on material and an intermediate barrier laminate, corresponding to the above explanation with reference to FIGS. 2 to 11.

The plunger 31 is again adapted as a syringe plunger and comprises three sealing lips 9.

FIGS. 16 and 17 show another embodiment of a plunger 32. Functions and components corresponding to those already explained above with reference to FIGS. 1 to 15 are designated with the same reference numbers and are not discussed again in detail.

The plunger 32 may again be configured with a stopper base body made of a base body material and a stopper overmold section made of an overmolding material, as explained above in connection with FIG. 1. Alternatively, or additionally, the plunger 32 may be configured with a stopper pre-mold body made of a pre-mold material, a stopper mold-on body made of a mold-on material and an intermediate barrier laminate, corresponding to the above explanation with reference to FIGS. 2 to 11.

The plunger 32 is again adapted as a syringe plunger and comprises two sealing lips 9.

FIGS. 18 and 19 show another embodiment of a plunger 33. Functions and components corresponding to those already explained above with reference to FIGS. 1 to 17 are designated with the same reference numbers and are not discussed again in detail.

The plunger 33 may again be configured with a stopper base body made of a base body material and a stopper overmold section made of an overmolding material, as explained above in connection with FIG. 1. Alternatively, or additionally, the plunger 33 may be configured with a stopper pre-mold body made of a pre-mold material, a stopper mold-on body made of a mold-on material and an intermediate barrier laminate, corresponding to the above explanation with reference to FIGS. 2 to 11.

The plunger 33 is again adapted as a syringe plunger and comprises two sealing lips 9.

FIGS. 20 and 21 show another embodiment of a plunger 35. Functions and components corresponding to those already explained above with reference to FIGS. 1 to 19 are designated with the same reference numbers and are not discussed again in detail.

The plunger 35 may again be configured with a stopper base body made of a base body material and a stopper overmold section made of an overmolding material, corresponding to the explanation above in connection with FIG. 1. Alternatively, or additionally, the plunger 35 may be configured with a stopper pre-mold body made of a pre-mold material, a stopper mold-on body made of a mold-on material and an intermediate barrier laminate, corresponding to the above explanation with reference to FIGS. 2 to 11. Two examples of the connection plane 19 are illustrated in FIG. 20. Other positions of the connection plane 19 between the stopper pre-mold body and the stopper mold-on body are also possible with a corresponding design of the plunger 35.

The plunger 35 is again adapted as a syringe plunger and comprises three sealing lips 9.

FIGS. 22 and 23 show another embodiment of a plunger 36. Functions and components corresponding to those already explained above with reference to FIGS. 1 to 21 are designated with the same reference numbers and are not discussed again in detail.

The plunger 36 may again be configured with a stopper base body made of a base body material and a stopper overmold section made of an overmolding material, as explained above in connection with FIG. 1. Alternatively, or additionally, the plunger 36 may be configured with a stopper pre-mold body made of a pre-mold material, a stopper mold-on body made of a mold-on material and an intermediate barrier laminate, corresponding to the above explanation with reference to FIGS. 2 to 11.

The plunger 36 is again adapted as a syringe plunger and comprises three sealing lips 9.

FIGS. 24 and 25 show another embodiment of a plunger 37. Functions and components corresponding to those already explained above with reference to FIGS. 1 to 23 are designated with the same reference numbers and are not discussed again in detail.

The plunger 37 may again be configured with a stopper base body made of a base body material and a stopper overmold section made of an overmolding material, as explained above in connection with FIG. 1. Alternatively, or additionally, the plunger 37 may be configured with a stopper pre-mold body made of a pre-mold material, a stopper mold-on body made of a mold-on material and an intermediate barrier laminate, corresponding to the above explanation with reference to FIGS. 2 to 11.

The plunger 37 is again adapted as a syringe plunger and comprises two sealing lips 9.

FIGS. 26 and 27 show another embodiment of a plunger 39. Functions and components corresponding to those already explained above with reference to FIGS. 1 to 25 are designated with the same reference numbers and are not discussed again in detail.

The plunger 39 may again be configured with a stopper base body made of a base body material and a stopper overmold section made of an overmolding material corresponding to the explanation above in connection with FIG. 1. Alternatively, or additionally, the plunger 39 may be configured with a stopper pre-mold body made of a pre-mold material, a stopper mold-on body made of a mold-on material and an intermediate barrier laminate, corresponding to the above explanation with reference to FIGS. 2 to 11.

The plunger 39 is again adapted as a syringe plunger and comprises three sealing lips 9.

FIGS. 28 and 29 show another embodiment of a plunger 40. Functions and components corresponding to those already explained above with reference to FIGS. 1 to 27 are designated with the same reference numbers and are not discussed again in detail.

The plunger 40 may again be configured with a stopper base body made of a base body material and a stopper overmold section made of an overmolding material, as explained above in connection with FIG. 1. Alternatively, or additionally, the plunger 40 may be configured with a stopper pre-mold body made of a pre-mold material, a stopper mold-on body made of a mold-on material and an intermediate barrier laminate, corresponding to the above explanation with reference to FIGS. 2 to 11.

The plunger 40 is again adapted as a syringe plunger and comprises two sealing lips 9.

FIGS. 30 and 31 show another embodiment of a plunger 41. Functions and components corresponding to those already explained above with reference to FIGS. 1 to 29 are designated with the same reference numbers and are not discussed again in detail.

The plunger 41 may again be configured with a stopper base body made of a base body material and a stopper overmold section made of an overmolding material, as explained above in connection with FIG. 1. Alternatively, or additionally, the plunger 41 may be configured with a stopper pre-mold body made of a pre-mold material, a stopper mold-on body made of a mold-on material and an intermediate barrier laminate, corresponding to the above explanation with reference to FIGS. 2 to 11.

The plunger 41 is again adapted as a syringe plunger and comprises two sealing lips 9.

In addition, the plunger 41 has a peg section 41a molded onto its front face facing the liquid 5, which, when the plunger 41 is fully extended, engages in a tip-side end of a syringe volume for discharging residual liquid.

FIGS. 32 and 33 show another embodiment of a plunger 42. Functions and components corresponding to those already explained above with reference to FIGS. 1 to 31 are designated with the same reference numbers and are not discussed again in detail.

The plunger 42 may again be configured with a stopper base body made of a base body material and a stopper overmold section made of an overmolding material, as explained above in connection with FIG. 1. Alternatively, or additionally, the plunger 42 may be configured with a stopper pre-mold body made of a pre-mold material, a stopper mold-on body made of a mold-on material and an intermediate barrier laminate, as already explained above with reference to FIGS. 2 to 11.

The plunger 42 is again adapted as a syringe plunger and comprises three sealing lips 9.

FIGS. 34 and 35 show another embodiment of a plunger 43. Functions and components corresponding to those already explained above with reference to FIGS. 1 to 33 are designated with the same reference numbers and are not discussed again in detail.

The plunger 43 may again be configured with a stopper base body made of a base body material and a stopper overmold section made of an overmolding material, as explained above in connection with FIG. 1. Alternatively, or additionally, the plunger 43 may be configured with a stopper pre-mold body made of a pre-mold material, a stopper mold-on body made of a mold-on material and an intermediate barrier laminate, corresponding to the above explanation with reference to FIGS. 2 to 11.

The plunger 43 is again adapted as a syringe plunger and comprises three sealing lips 9.

As in the embodiment shown in FIGS. 12 and 13, a residual discharge pen 30a is also an integral part of the plunger 43.

FIGS. 36 and 37 show another embodiment of a plunger 45. Functions and components corresponding to those already explained above with reference to FIGS. 1 to 35 are designated with the same reference numbers and are not discussed again in detail.

The plunger 45 may again be configured with a stopper base body made of a base body material and a stopper overmold section made of an overmolding material, as explained above in connection with FIG. 1. Alternatively, or additionally, the plunger 45 may be configured with a stopper pre-mold body made of a pre-mold material, a stopper mold-on body made of a mold-on material and an intermediate barrier laminate, corresponding to the above explanation with reference to FIGS. 2 to 5.

A receptacle 45a of the type of the receptacles 6a and 26 is, for the plunger 45, formed as a thread molded into the stopper base body or into the stopper pre-mold body or into the stopper mold-on body, respectively.

The plunger 45 is again adapted as a syringe plunger and comprises three sealing lips 9.

FIGS. 38 and 39 show another embodiment of a plunger 49. Functions and components corresponding to those already explained above with reference to FIGS. 1 to 37 are designated with the same reference numbers and are not discussed again in detail.

The plunger 49 may again be configured with a stopper base body made of a base body material and a stopper overmold section made of an overmolding material, as explained above in connection with FIG. 1. Alternatively, or additionally, the plunger 49 may be configured with a stopper pre-mold body made of a pre-mold material, a stopper mold-on body made of a mold-on material and an intermediate barrier laminate, corresponding to the above explanation with reference to FIGS. 2 to 11.

The plunger 49 is again adapted as a closure stopper and comprises two sealing lips 9.

FIGS. 40 and 41 show another embodiment of a plunger 50. Functions and components corresponding to those already explained above with reference to FIGS. 1 to 39 are designated with the same reference numbers and are not discussed again in detail.

The plunger 50 may again be configured with a stopper base body made of a base body material and a stopper overmold section made of an overmolding material, as explained above in connection with FIG. 1. Alternatively, or additionally, the plunger 50 may be configured with a stopper pre-mold body made of a pre-mold material, a stopper mold-on body made of a mold-on material and an intermediate barrier laminate, corresponding to the above explanation with reference to FIGS. 2 to 11.

The plunger 50 is again adapted as a closure stopper and comprises two sealing lips 9.

FIGS. 42 to 44 and FIGS. 45 to 47 show two more embodiments of the plunger 50 according to FIGS. 40 and 41 with the sealing lips 9 having different profile cross-sections. This profile cross-section of the sealing lips is in the embodiment of the plunger 50 according to FIGS. 40 and 41 approximately semicircular.

In the plunger 50 according to FIGS. 42 to 44, the profile cross-section is asymmetrical, with an axially outer conical section 9a and a convexly rounded axially inner profile section 9b (see the side view according to FIG. 44).

In the plunger 50 according to FIGS. 45 to 47, a radial projection of the sealing lips 9 beyond a cylindrical base body of the plunger 50 is slightly smaller than in the embodiment according to FIGS. 42 to 44. In addition, the two sealing lips 9 in the embodiment shown in FIGS. 45 to 47 are axially spaced further apart than in the embodiment shown in FIGS. 42 to 44.

FIGS. 48 and 49 show another embodiment of a plunger 51. Functions and components corresponding to those already explained above with reference to FIGS. 1 to 47 are designated with the same reference numbers and are not discussed again in detail.

The plunger 51 may again be configured with a stopper base body made of a base body material and a stopper overmold section made of an overmolding material, as explained above in connection with FIG. 1. Alternatively, or additionally, the plunger 51 may be configured with a stopper pre-mold body made of a pre-mold material, a stopper mold-on body made of a mold-on material and an intermediate barrier laminate, corresponding to the above explanation with reference to FIGS. 2 to 11.

The plunger 51 is again adapted as a syringe plunger and comprises three sealing lips 9.

FIGS. 50 and 51 show another embodiment of a plunger 52. Functions and components corresponding to those already explained above with reference to FIGS. 1 to 49 are designated with the same reference numbers and are not discussed again in detail.

The plunger 52 may again be configured with a stopper base body made of a base body material and a stopper overmold section made of an overmolding material, as explained above in connection with FIG. 1. Alternatively, or additionally, the plunger 52 may be configured with a stopper pre-mold body made of a pre-mold material, a stopper mold-on body made of a mold-on material and an intermediate barrier laminate, corresponding to the above explanation with reference to FIGS. 2 to 11.

The plunger 52 is again adapted as a syringe plunger and comprises two sealing lips 9.

FIGS. 52 and 53 show another embodiment of a plunger 53. Functions and components corresponding to those already explained above with reference to FIGS. 1 to 51 are designated with the same reference numbers and are not discussed again in detail.

The plunger 53 may again be configured with a stopper base body made of a base body material and a stopper overmold section made of an overmolding material, as explained above in connection with FIG. 1. Alternatively, or additionally, the plunger 53 may be configured with a stopper pre-mold body made of a pre-mold material, a stopper mold-on body made of an mold-on material and an intermediate barrier laminate, corresponding to the above explanation with reference to FIGS. 2 to 11.

The plunger 53 is again adapted as a syringe plunger and comprises two sealing lips 9.

With regard to the manufacturing method for producing the plungers 30 to 53, the above explanations relating to the plungers 3, 11, and 25 apply.

1. A medical or pharmaceutical stopper for sealing a container volume of a container for receiving a liquid for medical, pharmaceutical, or analytical use,
comprising a stopper base body made of a base body material,
comprising a stopper overmold section made of an overmolding material, via which the stopper can be sealingly placed on an inner wall of the container.

2. The medical stopper according to claim 1, wherein the base body material is a thermoplastic.

3. The medical stopper according to claim 2, wherein the base body material is at least one of the following materials: PC, PMMA, PBT, PA, PEEK.

4. The medical stopper according to claim 1, wherein the base body material is silicone.

5. The medical stopper according to claim 1, wherein the overmolding material is silicone.

6. The medical stopper according to claim 1, wherein the stopper base body and the stopper overmold section are mechanically connected to one another.

7. The medical stopper according to claim 1, wherein the stopper base body and the stopper overmold section are chemically connected to one another.

8. The medical stopper according to claim 1, wherein stopper base body comprises a receptacle for a plunger rod.

9. The medical stopper according claim 1, wherein the stopper overmold section comprises:
a stopper pre-mold body made of a pre-mold material,
a stopper mold-on body made of a mold-on material,
a barrier laminate made of a laminate material, which is laminated onto the stopper pre-mold body and at least partially overmolded by the stopper pre-mold body and the stopper mold-on body and is arranged between the stopper pre-mold body and the stopper mold-on body, the barrier laminate covering at least 30% of a sealing cross-section of the stopper.

10. The method for manufacturing a medical stopper according to claim 9, comprising the following steps:
manufacturing the stopper pre-mold body,
manufacturing the barrier laminate,
positioning the barrier laminate by placing it on the stopper pre-mold body,
injection-molding the stopper mold-on body onto the stopper pre-mold body such that the barrier laminate is at least partially overmolded by the stopper pre-mold body and the stopper mold-on body during the mold-on process.

11. The method according to claim 10, wherein the barrier laminate is completely overmolded when molding the stopper mold-on body onto the stopper pre-mold body.

12. The method according to claim 10, wherein tempering the medical stopper after the mold-on process.

13. The method according to claim 10, wherein the overmolding material is silicone.

14. The medical stopper according to claim 9, wherein a radially outer section of the barrier laminate is angled like a truncated cone section.

15. The medical stopper according to claim 14, wherein the base body material is silicone.

16. The medical stopper according to claim 14, wherein the overmolding material is silicone.

17. The medical stopper according to claim 14, wherein the stopper base body and the stopper overmold section are mechanically connected to one another.

18. The medical stopper according to claim 14, wherein the stopper base body and the stopper overmold section are chemically connected to one another.

19. The medical stopper according to claim 14, wherein the stopper base body comprises a receptacle for a plunger rod.
